(19) United States
(12) Patent Application Publication
(10) Pub. No.: US 2026/0216448 A1
(43) Pub. Date: Jul. 30, 2026

(54) IMPROVEMENTS IN AND RELATING TO MEDICAL NEEDLE COVER ARRANGEMENTS

(71) Applicant: TIP-TOP.COM LTD, Colchester (GB)

(72) Inventor: Barry P. Liversidge, Colchester (GB)

(73) Assignee: TIP-TOP.COM LTD, Colchester (GB)

(21) Appl. No.: 19/140,703

(22) PCT Filed: Dec. 20, 2023
(86) PCT No.: PCT/GB2023/053327

§ 371 Date: Jun. 18, 2025

(30) Foreign Application Priority Data
Dec. 23, 2022 (GB) 2219711.5
Apr. 6, 2023 (GB) 2305213.7

(51) Int. Cl. A61M 5/32

(52) U.S. Cl. CPC A61M 5/3216; A61M 5/3202

(57) ABSTRACT

The present invention relates to a medical needle cover arrangement comprising a safety device (8) comprising a needle shield (19) pivotally coupled to a carrier (18), wherein the carrier (18) is configured to be secured to the syringe (20), and the needle shield (19) is configured to move outwardly from an initial shielding position to a non-shielding position. A needle cover (23) protects the medical needle (11). In an initial shielding position, the needle cover (23) is located within the needle shield (19) at a first position and a part of the needle cover (23), and a part of the needle shield (19) are configured to engage when the needle shield (19) is at the initial shielding position to releasably maintain the needle cover (23) within the needle shield (19) at said first position. The part of the needle shield (19) engaging the part of the needle cover (23) prevents distal movement of the needle cover (23) relative to the needle shield (19) away from the first position until the needle shield 19 is moved to the non-shielding position. The needle shield (19) is arranged to move outwardly away from the needle cover (23) and is configured to be manipulated away from the initial shielding position to the non-shielding position. This movement of the needle shield (19) disengages the needle shield (19) from the needle cover (23) to thereafter allow subsequent detachment of the needle cover (23) to uncover the medical needle (11).

### FIELD OF THE INVENTION

This invention relates to a medical needle cover arrangement for a needle cover used in association with a syringe having a medical needle projecting forwardly therefrom. In its preferred aspects, this invention concerns improvements in a needle cover associated with a needle safety device arranged to confer needlestick protection to a medical needle projecting forwardly from a single-use syringe.

### BACKGROUND TO THE INVENTION

A syringe provided with a medical needle as employed in this invention is intended to be used to penetrate a human or animal body, or for other medical uses such as the penetration of a pierceable membrane of an intravenous medication system. In the following all medical uses of the syringe and needle will be described simply as the penetration of a body, even though specific embodiments may be intended for other medical uses.

A syringe having a needle permanently secured thereto is frequently pre-filled with a liquid drug or medicament and then is used only once to perform an injection. Once used, the syringe and needle must be disposed of in a safe manner. To protect any people who might have to handle such a syringe, either before or after performing an injection, it is becoming a requirement of health and safety legislation as well as best practice to provide the needle with some kind of safety device to minimise the risk of accidental needlestick injury.

Typically, such a safety device may have a shield which is mounted on the syringe and is slidable axially between a needle protecting position and a non-protecting position.

In the course of manufacture of a single-use syringe having a needle permanently secured to a needle hub at the forward end of the syringe barrel, it is the usual practice to fit a needle cover over the needle before the syringe is packaged and then sterilised for storage and transport, prior to filling with a liquid drug or other medicament. Such a cover is usually made of a soft elastomer such as rubber or a TPE and must be compliant with drug compatibility and stability. The rear end of the cover fits onto the needle hub and the sharp tip of the needle penetrates the material of the cover so as to be sealed thereby, but the cover does not bear on the greater part of the length of the needle so as not to remove any lubricant which is usually be applied to the needle. Thus, there is a void around the greater part of the needle, from the hub of the syringe to a location adjacent the sharp tip of the needle. This part of the needle must be maintained in a sterile environment prior to use of the needle. EP 1502617 and EP 1964586 are examples of needle shields that have a rigid outer shield which cover a soft inner (elastomeric or rubber) needle cover, generally as described above.

A significant problem associated with the manufacturing process for a single-use syringe is that in the course of the packaging and sterilisation steps, there is a tendency for a needle cover to be displaced from its as-fitted condition on the hub-a phenomenon known as “pop-off”. This primarily occurs on account of the changes of pressure to which the syringe and cover are subjected in the course of the sterilising process and the fact that the void between the needle and the cover is in effect a sealed space. The problem has been addressed by venting that space, but then there is a possibility that the sterile condition of the needle may be compromised. As a consequence, in an attempt to prevent “pop-off” by increasing the security of the attachment of a needle cover to the hub of a syringe during the sterilisation process, it is the usual practice to provide at least one formation on the hub of the syringe and a corresponding formation at the rear end of the needle cover to co-operate with the formation on the hub, to mechanically hold the needle cover on the syringe hub. For example, an upstanding rib may be formed around the hub, the needle cover having an in-turned flange at its rear end, and which is fitted over the rib to engage there behind and so hold the cover on to the syringe hub in a fixed position.

As mentioned above, the needle must be sterilised and then subsequently maintained within the cover to keep it sterilised and this cover must therefore be removed prior to use. Accordingly, as may be the case with a needle cover associated with a needle safety device having a protective shield arranged to confer needlestick protection, the needle cover may be located within the protective shield so that an end distal portion of the cover projects beyond the distal end of the protective shield. The user then firstly grasps the end portion and to withdraw the cover from within the shield before the injection can be performed. By way of example EP 2533835 discloses a medical needle cover arrangement which is used commercially within the pharmaceutical industry, and wherein the furthermost distal end portion of the cover extends through an opening at the furthermost distal end of a telescopic needle shield. The needle shield is telescopically mounted to a carrier such that the needle shield can be retracted proximally to expose the needle. To allow for this telescopic proximal movement, the shield is located around an outer circumference of a distal end portion of the syringe which increases the overall diameter of the assembly relative to the diameter of the syringe. As mentioned above, the furthermost distal end portion of the cover must extend beyond the furthermost distal end of the needle shield, and because the length of the needle shield must of course be of sufficient length to shield the entirety of the needle, it follows that the length of the needle cover must be additionally longer than needle shield, to allow for this distal extension of the cover to project past the end of the shield, to enable a user to easily grasp the cover to pull it distally forward and out from within the rigid needle shield before the performing an injection.

Therefore as described in EP 2533835 the needle shield has to be of an overall diameter greater than the overall diameter of the syringe barrel. In addition, to enable a user to grip and remove the cover from within the shield the needle cover has to be considerably longer than a typical standard needle cover described in EP 1502617 and EP 1964586.

Automated methods and systems are used to fill pre-fillable medical syringes, many of which use a tray (nest) and tub arrangement. Such nest and tub arrangements utilise standard size trays (which typically accommodate 160 ready-to-fill syringes per tray and each tray is housed within a standard depth tub) which help to automate standard universal syringe filling systems. However, the device described in EP 2533835 and many safety needle devices integrated onto prefillable syringes cannot be accommodated into a standard nest and tub system without major adaptations to both the tray/nest and the tub. In fact, due to the increased bulk of some designs, only 100 quantity syringes can be fitted into each tray instead of the usual 160 quantity, which not only creates problems within the automated syringe filling systems, but also greatly increases bulk storage of the packaged syringes per pallet.

This invention is suitable for integration onto entirely standard syringes and does not require any modification to either the tray/nest or the tub used within these systems. The sterilisation process may be undertaken whilst the syringes are packaged and contained on pallets and boxes and within the nest and tub arrangements, and so accordingly, the use of standard nest and tub systems can be achieved rather than bespoke modified systems.

To enable incorporation into standard nest (tray) and tub formats, it is a principal aim of the present invention to minimise the problems discussed above of providing a needle cover on a single-use pre-filled syringe having a needle secured to the forward end thereof and in particular to prevent the phenomenon of “pop-off” associated with the manufacture of conventional sterile ready-to-fill (pre-fillable) syringes and to provide a product that is suitable for the associated automatic assembly, filling and inspection lines; and ensure the needle cover is safely secured during storage of the prefilled syringe.

It is an aim of the present invention to overcome at least one problem associated with the prior art whether referred to herein or otherwise.

### SUMMARY OF THE INVENTION

According to a first aspect of the present invention there is provided a medical needle cover arrangement comprising:
a syringe having a syringe barrel including a needle hub at the distal end thereof, the syringe further comprising
a medical needle having a longitudinal axis and a sharp tip, the medical needle being mounted on the needle hub with the sharp tip projecting distally therefrom;
a safety device comprising a needle shield pivotally coupled to a carrier, wherein the carrier is configured to be secured to the syringe, and the needle shield is configured to move outwardly (relative to the needle axis) from an initial shielding position to a non-shielding position;
a needle cover overlying the medical needle to protect the needle and prevent drug leakage from the sharp tip;
the needle cover comprises,
   a longitudinal axis,
   a profile at a proximal end configured to engage a profile on a distal end of the needle hub to create a substantially airtight seal therebetween to maintain the sterility of the enclosed medical needle within the needle cover when the needle cover is located at a first position relative to the needle hub; and
in the initial shielding position, the needle cover is located within the needle shield at said first position, whereat the longitudinal axis of the needle cover is aligned with the longitudinal axis of the needle;
characterised in that:
the needle shield comprises clasping means to clasp and/or grip or loosely hold and retain the needle cover in the first position; and wherein
the needle shield, the carrier and the needle cover are maintained together as a unitary self-supporting safety needle assembly for attachment onto the distal end of the syringe;
whereat a part of the needle cover and a part of the needle shield are configured to engage when the needle shield is at the initial shielding position to releasably maintain the needle cover within the needle shield at said first position to prevent drug leakage and maintain the sterility of the medical needle during storage of the syringe;
the part of the needle shield engaging the part of the needle cover blocks distal movement of the needle cover relative to the needle hub distally away from the first position when the safety needle assembly is attached to the distal end of the syringe until the needle shield is moved to the non-shielding position; and wherein
the needle shield is arranged to move outwardly away from the needle cover and is configured to be manipulated away from the initial shielding position to the non-shielding position; said movement of the needle shield away from the initial shielding position disengages the needle shield from the needle cover to thereafter allow (subsequent) detachment of the needle cover distally away from said first position to uncover the medical needle.

Preferably the clasping means comprise retaining clasps comprising shaped lugs having inner arcuate surfaces to locate around a part of an outer circumferential surface of the cover to grip or loosely hold the needle cover.

Preferably the clasping means provides a restraining force which must be overcome in order to move the needle shield outwardly from the initial shielding position to the non-shielding position.

The needle shield may comprise a longitudinally extending gap. The needle shield may comprise a partial cylindrical (tubular) shield in which a longitudinal gap is provided along at least a portion of the longitudinal length and preferably for the full longitudinal length of the needle shield.

The clasping means may comprise one or more clasps provided on or adjacent to edges of the longitudinal gap of the needle shield.

The unitary self-supporting safety needle assembly may be arranged to be press-fitted onto the syringe.

Preferably the engagement of the needle shield and needle cover during the attachment of the safety device on to the syringe introduces a compressive axial force in the distal direction in the needle cover, between a rear end of the needle cover and a location where the needle shield engages the needle cover.

Preferably the (furthermost) distal end of the needle cover is located (entirely) within the needle shield when the needle shield is in the initial shielding position and the needle cover is in the first position.

Preferably the (furthermost) distal end of the needle cover projects/protrudes past the (furthermost) distal end of the needle shield when the needle shield is in the initial shielding position and the needle cover is in the first position.

Preferably the needle shield is pivotally coupled to the carrier.

The needle shield may be secured to the carrier by a hinge arrangement/mechanism. The hinge mechanism may define a fixed rotational axis about which the needle shield rotates relative to the carrier. The fixed rotational axis may provide a revolute joint between the needle shield and the carrier.

Preferably the needle shield is arranged to pivot from the initial shielding position to a non-shielding position.

The needle shield may comprise two pivot member which may be engaged within two pivot members on the carrier. The needle shield may comprise two stub axles which may be engaged within two corresponding apertures on the carrier. The pivot members may define a pivoting axis for the needle shield.

The carrier may comprise two pivot member which may be engaged within two pivot members on the needle shield. The carrier may comprise two stub axles which may be engaged within two corresponding apertures on the needle shield. The pivot members may define a pivoting axis for the needle shield.

The needle shield may be arranged to move, and preferably pivotally move, and more preferably sequentially/successively move between:
the (before use) initial shielding position;
a (during use) non-shielding position; and
a (after use) final shielding position.

The needle shield may be arranged to move, and preferably pivotally move, and more preferably sequentially/successively move between:
a storage position (before use);
an operative position (during use); and
a safety position (after use).

The protective shield and the needle cover may form an interlock. Preferably the interlock provides a mechanism for co-ordinating the function of the protective shield and/or the needle cover. Preferably the interlock co-ordinates the function and/or relationship of the protective shield and/or needle cover in (each of) the sequential positions. Preferably, in the storage/initial shielding (before use) position, the interlock prevents access to distally remove the needle cover from within the protective shield.

In the final (safety) position, the sharp tip of the medical needle may be deflected away from the longitudinal axis of the syringe barrel. In the final shielding (safety) position, the medical needle may be deformed and preferably is non-linear. Preferably with this deformation the medical needle is not re-usable.

As the needle shield is moved from the non-shielding position to the final shielding (safety) position, an interior surface of the needle shield is arranged to contact a part (preferably the sharp tip) of the medical needle. Preferably this contact and the further movement of the needle shield causes the needle shield to subsequently deflect the medical needle.

The deformed medical needle may resiliently urge the needle shield outwardly back towards a non-shielding position.

The needle shield may comprise a resiliently deformable (elastomeric/resilient) locking region, zone or portion to lock the needle shield relative to the carrier in the final shielding position. The locking portion may comprise a proximal region, zone or portion of the needle shield.

The carrier may comprise a locking abutment surface to lock the needle shield relative to the carrier in the final shielding position.

The abutment surface may be provided on a part of the carrier.

The abutment surface may be provided on the carrier as a lug which preferably extends in a distally direction forwardly (outwardly) from the carrier.

The locking portion or region of the needle shield may engage with the abutment surface in the final shielding (safety) position. A sliding surface of the locking portion may be arranged to travel over a sliding surface of the carrier or the lug when moving from the non-shielding position to the final shielding position. The locking portion and/or the lug may resiliently deform as the locking portion moves over the distal end of locking abutment surface of the lug. In the final shielding position, the locking portion and the abutment surface of the lug are in an undeformed and unstressed (relaxed) condition/state.

In the final shielding position, the locking portion and the abutment surface may be resiliently engaged due to the flexural distortion of the deformed medical needle.

The needle cover may comprise a soft unitary component comprising an elastomeric plastic polymer or rubber material. The needle cover may comprise a component having an internal soft portion to enable the sharp tip of the needle to penetrate into the soft portion. The needle cover may also comprise a rigid portion. The rigid portion may comprise an outer rigid shell. The outer rigid shell may extend around a part or substantially all of an outer surface of the internal soft portion. The internal soft portion may comprise one or more rigid portions secured to an outer surface. The soft portion and the rigid portion may be co-moulded and/or bonded and adhered together, or be independent separate items.

A part of the needle cover may comprise a step or recess or outer projection or annular flange which may be configured to engage a part of the needle shield which may comprise a (rear/proximally facing) shoulder or inward projection in order to (releasably) engage with and maintain and hold the needle cover over the needle at the first position. Preferably at the first position, the engagement may cause a proximal portion of the needle cover to compress and/or expand due to engaged contact with a portion of the syringe hub. Preferably the step or recess may be located and spaced from a proximal distal end of the needle cover. Preferably a majority of the needle cover is encompassed by the needle shield in the initial shielding position when the needle cover is at the first position to prevent user-access to the cover to prevent the cover from being manually grasped and manipulated by the finger(s) of a user. In some embodiments the step or recess may be provided at the extreme furthermost distal end, or the step or recess may actually be formed by a distal end face of the needle cover. In other embodiments there may be no step or recess and the needle shield may frictionally grip and retain the needle cover over the needle at the first position.

The needle cover may be (purely/solely) frictionally engaged and/or gripped by the needle shield in order to maintain and hold the needle cover at the first position. An outer surface of the needle cover may be even (planar) and/or substantially smooth and this outer surface may be frictionally gripped by the needle shield. The needle shield may provide a correspondingly (planar) and/or substantially smooth inner surface in order to grip the needle cover. Preferably the movement of the needle shield away from the initial shielding position fictionally disengages the needle shield from the needle cover.

The carrier may comprise an annular ring component. The carrier may comprise a securement member to secure the carrier (and the safety device) to the syringe. The securement member may comprise an internal rib or a number of external ribs which may be secured to a (rear facing) shoulder provided on the hub of the syringe. The internal rib may comprise a circumferential rib on an inner surface of the carrier and may extend around or partly around the inner circumferential surface of the ring component to encapsulate the needle hub. A part, and preferably an arcuate part of the internal rib may be resiliently movable outwardly to enable a (rear facing) shoulder on the needle hub to pass the internal rib such that the internal rib flexes outwardly and then resiliently return towards an original/initial state to thereafter prevent removal of the syringe from the carrier.

Preferably the part of the needle shield engages the needle cover at a location spaced distally forwardly from a rear proximal end thereof when the needle shield is in the initial shielding position to resist movement of the needle cover forwardly and distally away from the syringe hub. The needle shield may encompass a circumferential portion of the outer surface of the needle cover and may extend along the entire longitudinal length of the needle cover, and the needle shield may extend distally to a position beyond the most distal point of the needle cover such that the entire needle cover is disposed within the longitudinal length of the needle shield.

The needle cover may be made from a resilient material and be resiliently compressible and expandible in an axial direction and resiliently compressible and expandable in a radial direction.

The length of the needle cover from a rear proximal face to the engagement location within the needle shield, is such that by fitting the carrier and the needle shield and needle cover to the syringe, a (rearward) proximal portion of the needle cover is subjected to an axial compression force to cause an internal profile of the rearward proximal portion of the needle cover to expand resiliently outwards to fit onto a forward distal part of the syringe hub, thereby ensuring a substantially airtight seal between the resiliently urged proximal end face of the needle cover and the needle hub of the syringe. The axial compression force may cause a profile of the rearward proximal portion of the needle cover to enragedly compress against a forward distal part of the syringe hub, thereby ensuring a substantially airtight seal between the resiliently urged proximal end face of the needle cover and the needle hub of the syringe.

Preferably a substantially airtight seal is formed between the rear proximal end of the needle cover and the forward distal end of the syringe hub by virtue of the axial force and the correspondingly profiled surfaces on the distal end of the syringe hub acting to cause elastic expansion of the rearward end of the needle cover as it is forced onto the syringe hub when the safety device is fitted to the syringe. Preferably, so long as a safety device including a needle shield for the medical needle is fitted to the syringe to provide the engagement between the needle cover and the needle shield, there is no need for the needle cover to be held on to the needle hub by mechanical inter-engagement of formations on the needle hub corresponding to inter-engagement formations on the needle cover as is utilised in standard needle covers described in EP 1502617 and EP 1964586, such as an in-turned flange which engages behind a rib formed around the bulbous hub of the syringe. Preferably, in this invention there is no dependency or requirement for the mechanical inter-engagement formations currently relied upon by the industry standard needle covers used in the market today. Instead, the seal of this invention may be formed forwardly of any rib or other formation provided on the needle hub of the syringe. Preferably the engagement between the needle cover and the needle shield when in the initial shielding position serves to prevent the cover sliding distally forward off the needle hub of the syringe from the first position during the manufacturing and sterilisation process or subsequently during storage of the syringe, by virtue of the fact that the needle shield is restrained against pivotal movement away from the initial shielding position.

The clasping means may comprise one or more clasps provided on the needle shield and preferably on edges (or adjacent to edges) of a longitudinal gap of the needle shield. The clasping means may provide a restraining force which must be overcome in order to move the needle shield outwardly from the initial shielding position to the non-shielding position. The clasping means (preferably together with the pivotable coupling/hinge mechanism arrangement) may form a sub-assembly comprising the carrier, the needle shield and the needle cover and wherein this sub-assembly is maintained and held together as a complete unitary assembly and is preferably self-supporting, due to the restraining force provided by clasps and or because the needle cover is otherwise held by frictional engagement within the needle shield.

The engagement of the needle shield and needle cover during the attachment of the safety device on to the syringe introduces a compressive axial force in the distal direction in the needle cover, between a rear end of the needle cover and a location where the needle shield engages the needle cover. This may urge a/the resilient proximal region of the needle cover rearwardly and proximally relative to the syringe, to ensure a sterile seal is maintained between the rear end of the needle cover and the needle hub. In view of the resilient nature of the needle cover, the compressive axial force in a distal direction applied to the needle cover may be maintained during manufacture, transport and storage of the syringe, so in turn ensuring that an effective seal is maintained, even during the sterilisation process which usually subjects the closed void/space around the needle to differential atmospheric pressure which in turn generates a resultant axial force in the needle cover away from the needle hub in a distal direction.

As with a conventional needle cover, the sharp tip of the medical needle may be received in a (resilient/soft/elastomeric) portion of the needle cover so as to be sealed thereby. In addition, this confers further protection to the sharp tip of the medical needle in the various stages of the manufacture of the syringe, its sterilisation, packaging, subsequent unpacking for filling, filling and re-packaging.

According to a second aspect of the present invention there is provided a method of protecting a medical needle comprising providing a medical needle cover arrangement which comprises:
a syringe having a syringe barrel including a needle hub at the distal end thereof,
the syringe further comprising the medical needle having a longitudinal axis and a sharp tip, the medical needle being mounted on the needle hub with the sharp tip projecting distally therefrom;
a safety device comprising a needle shield pivotally coupled to a carrier, wherein the carrier is configured to be secured to the syringe, and the needle shield is configured to move outwardly from an initial shielding position to a non-shielding position;
a needle cover overlying the medical needle to protect the needle and prevent drug leakage from the sharp tip;
the needle cover comprises,
   a longitudinal axis,
   a profile at a proximal end configured to engage a profile on a distal end of the needle hub to create a substantially airtight seal therebetween to maintain the sterility of the enclosed medical needle within the needle cover when the needle cover is located at a first position relative to the needle hub;
in the initial shielding position, the needle cover is located within the needle shield at said first position, whereat the longitudinal axis of the needle cover is aligned with the longitudinal axis of the needle;
characterised in that:
the needle shield comprises clasping means to clasp and/or grip or loosely hold and retain the needle cover in the first position; and wherein the needle shield, the carrier and the needle cover are maintained together as a unitary self-supporting safety needle assembly for attachment onto the distal end of the syringe;
whereat a part of the needle cover and a part of the needle shield are configured to engage when the needle shield is at the initial shielding position to releasably maintain the needle cover within the needle shield at said first position to prevent drug leakage and maintain the sterility of the medical needle during storage of the syringe;
the method comprising:
blocking distal movement of the needle cover relative to the needle hub distally away from the first position when the safety needle assembly is attached to the distal end of the syringe until the needle shield is moved to the non-shielding position by engaging the part of the needle shield with the part of the needle cover; and wherein
the needle shield is arranged to move outwardly away from the needle cover and is configured to be manipulated away from the initial shielding position to the non-shielding position; said movement of the needle shield away from the initial shielding position disengages the needle shield from the needle cover to thereafter allow (subsequent) detachment of the needle cover distally away from said first position to uncover the medical needle.

Preferably, in use, the needle shield is first/initially moved outwardly whilst the needle remains enclosed within the needle cover and, once the needle shield is in the outward non-shielding position, the needle cover can then be grasped by a user and moved distally and detached from the syringe to uncover the needle in preparation for an injection. Accordingly, the needle shield prevents “user access” to the needle cover to prevent the cover from being removed whilst the needle shield is in the initial shielding position and only/solely allows the needle cover to be removable whilst in the non-shielding position, just prior to performing an injection. In this way, the needle cover may remain at the first position until the needle shield is moved to the non-shielding position at which point a user is able to decide when to grasp and remove the needle cover, thus keep the needle protected until ready to perform an injection. For example, there may be a period of time between moving the needle shield to the non shielding position and the detachment of the needle cover from the syringe.

### BRIEF DESCRIPTION OF THE DRAWINGS

The present invention will now be described, by way of example only, with reference to the drawings that follow, in which:

FIG. 1 is a perspective view of a first preferred embodiment of a safety needle assembly and a syringe prior to attachment together;

FIG. 2 is a perspective view of the first preferred embodiment of the safety needle assembly attached to the syringe;

FIG. 3 is a perspective view of the first preferred embodiment of the safety needle assembly attached to the syringe with the protective shield in a non-shielding position and a cover removed;

FIG. 4 is a perspective view of the first preferred embodiment of the safety needle assembly attached to the syringe after an injection with the protective shield in a final shielding position;

FIG. 5 is a perspective view of the first preferred embodiment of the safety needle assembly prior to attachment to a syringe;

FIG. 6 is a side view of the first preferred embodiment of the safety needle assembly prior to attachment to a syringe;

FIG. 7 is a top plan view of the first preferred embodiment of the safety needle assembly prior to attachment to a syringe;

FIG. 8 is a side cross section of the first preferred embodiment of the safety needle assembly and a side view of the syringe prior to attachment together;

FIG. 9 is a side cross section of the first preferred embodiment of the safety needle assembly and a side view of the syringe attached together;

FIG. 10 is a side cross section of the first preferred embodiment of the safety needle assembly and a side view of the syringe attached together with the protective shield moving towards a non-shielding position;

FIG. 11 is a side cross section of the first preferred embodiment of the safety needle assembly and a side view of the syringe attached together with the protective shield in a non-shielding position and the cover removed from the syringe;

FIG. 12 is a side cross section of the first preferred embodiment of the safety needle assembly and a side view of the syringe attached together with the protective shield moving towards the final shielding position;

FIG. 13 is a side cross section of the first preferred embodiment of the safety needle assembly and a side view of the syringe attached together with the protective shield in the final shielding position;

FIG. 14 is a detailed view of a side cross section of the first preferred embodiment of the safety needle assembly and a side view of the syringe attached together showing the proximal region of the protective shield in an unlocked position relative to the locking projection;

FIG. 15 is a detailed view of a side cross section of the first preferred embodiment of the safety needle assembly and a side view of the syringe attached together with the protective shield in the final shielding position showing the proximal region of the protective shield in a locked position relative to the locking projection;

FIG. 16 is a side cross section of a further preferred embodiment of the safety needle assembly and a side view of the syringe attached together; and

FIG. 17 is a side cross section of the further preferred embodiment of the safety needle assembly and a side view of the syringe attached together with the protective shield in a non-shielding position and the cover removed from the syringe.

### DESCRIPTION OF THE PREFERRED EMBODIMENTS

Throughout this specification and with reference to the figures, a safety needle assembly or safety device 8 is shown and described herein which provides for shielding of a sharp tip of a needle 11 of a syringe. As used herein, the term “distal” and/or “forwards” or“ forwardly”, and derivatives thereof, refer to the direction generally towards the patient end for use, and the term “proximal” and/or “rearwards” or “rearwardly”, and derivatives thereof, is used to describe the direction away from the patient during use. As shown in the figures and as will be described, the proximal end of the safety needle assembly attached to a distal end of a syringe 10.

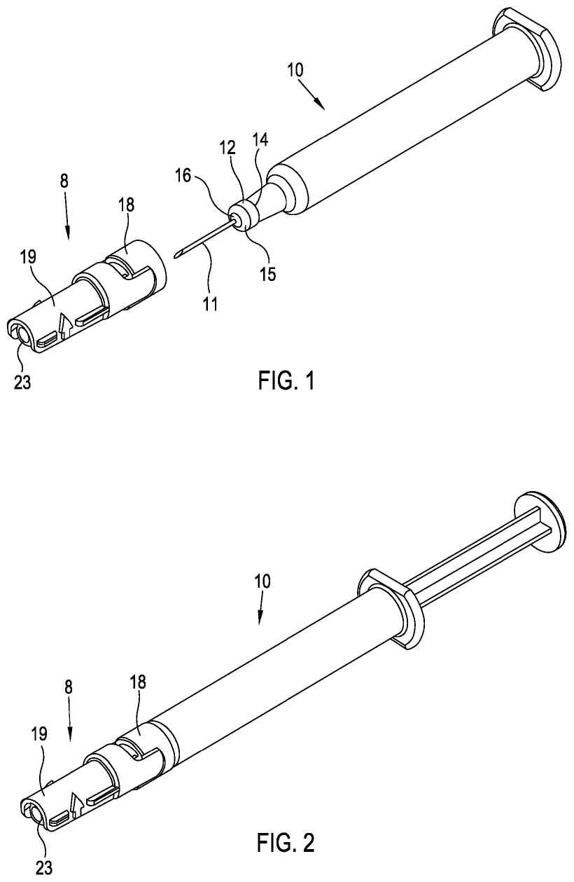

Referring initially to FIGS. 1 to 13, there is shown a syringe 10 having a needle 11 staked-in to the nose 12 of the syringe 10 and secured by adhesive, and a safety device 8 to confer protection on the needle 11. The nose 12 of the syringe 8 serves as a hub for the needle 11 and defines a rearwardly facing shoulder 14 and forwardly of that shoulder 14 the nose 12 is provided with a bulbous profile which defines an engagement face 15. Centrally of the nose 12, a small mass of adhesive 16 serves to secure the needle 11 in a bore extending through the nose 12. The safety device 8 includes a carrier 18 in the form of a tubular body or sleeve section for a protective shield 19 (needle shield), movable (specifically, pivotable/tiltable/rotatable) with respect to the longitudinal axis of needle 11, when an injection is to be performed. The carrier 18 has a bore 20 for the nose 12 of the syringe 10, an inwardly directed rib 21 being formed in the bore 20 for engagement behind the shoulder 14 when the safety device 8 is fitted to the syringe 10 so as to hold the safety device 8 to the syringe 10.

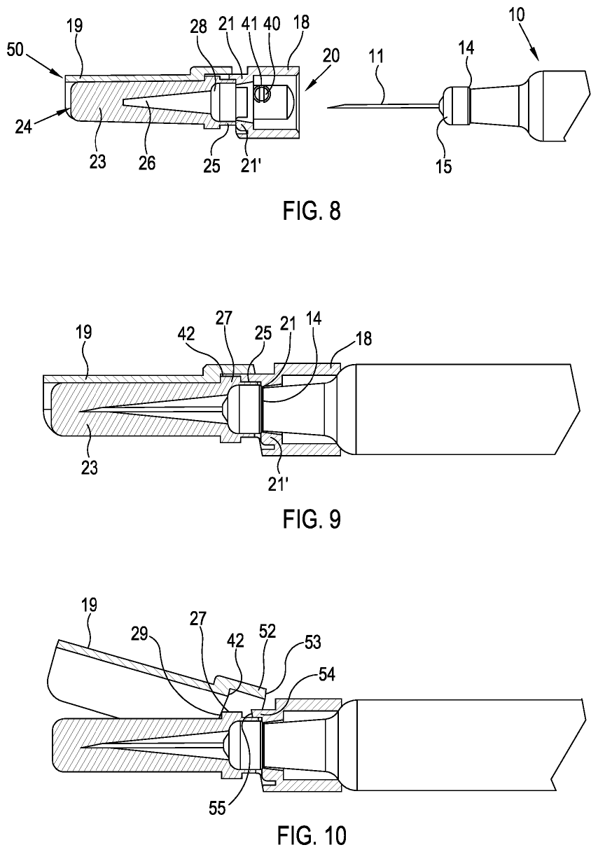

The rib 21 may comprise at least a section of an outwardly flexible rib 21′ (as shown in FIG. 8) which will flex outwardly during the insertion of the nose 12 into the bore 20. This flexible section 21′ includes a sloped inward abutment surface for engagement with the outer circumferential surface of the nose 12. The translational movement of the nose 12 into the bore 20 thereby causes the outer surface of the flexible section 21′ to be deflected outwardly until the shoulder 14 projects distally beyond the end of the flexible section 21′. At this position, the flexible section 21′ will snap back inwardly to engage the carrier 18 to the nose 12. The corresponding shapes of the shoulder 14 and the distal surface of the rib 21 and flexible section 21′ prevent the nose 12 from being withdrawn from the bore 20 in this engaged position. Accordingly, this provides a secure single use snap lock permanent fit for the safety assembly 8 to the syringe 10.

The protective shield 19 is arranged to securely maintain the position of a (soft rubber) cover 23 within the safety device 8.

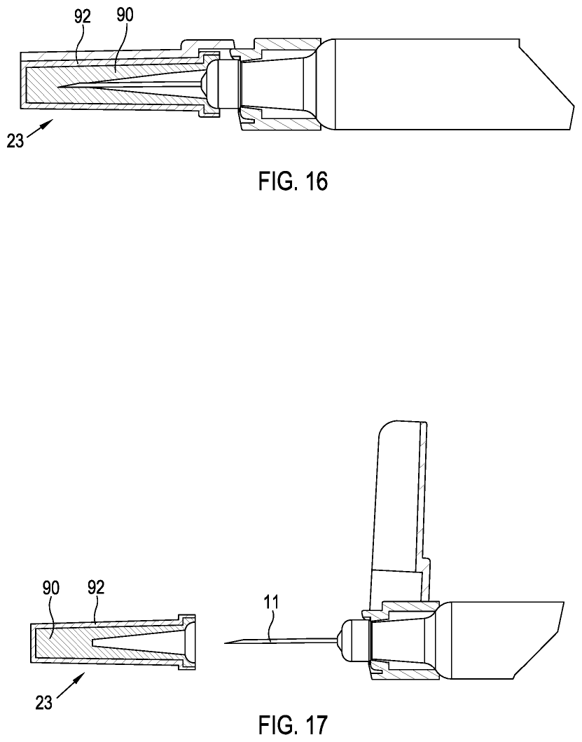

Some of the embodiments are shown in which the needle cover 23 comprises a soft needle cover. The soft needle cover may be entirely made of a soft material or may comprise just a portion/part made of a soft material. It should be readily appreciated that the needle cover 23 enables the sharp tip of the needle 11 to penetrate partially into an internal soft surface to allow the functionality of the needle cover 23 and other parts/surfaces of the needle cover 23 may not be soft and may in fact be more rigid than the soft part. For example, as shown in FIG. 16 and FIG. 17, the needle cover 23 may have a rigid outer shell 92 located over or provided around an outer surface or a part/parts of an outer surface of an internal soft portion 90. The internal soft portion 90 may be defined in its own right as a soft needle cover albeit, in examples, there is a full or partial outer rigid shell 92 used in combination. This rigid outer shell 92 or a similar rigid component(s) may be secured around a part or parts of the internal soft component 90 in any suitable configuration. Such a combination of parts 90, 92 still fall within the definition of a soft needle cover since the essential internal part of the needle cover provides a soft material to allow penetration of the sharp tip of the needle 11. Accordingly, the term soft needle cover may be used even though it is not a requirement that each and every portion of the needle cover 23 is soft.

Typically, the soft needle cover or component 90 may be a push-fit into the outer shell 92 during the manufacturing process. Alternatively, the outer shell 92 and soft needle component 90 may be co-moulded (also known as over-moulding) and both bonded together during a plastic injection manufacturing process. The outer shell/cover may cover only a part of the soft needle cover, for instance only at the rearward proximal end. The harder outer shell 92 may provide a more slippery low friction surface to enable a clasp arrangement 44 of the needle shield 19 to more easily disengage from the needle cover 23, when the shield 19 is manipulated away from the initial shielding position.

As mentioned above, the needle cover may comprise a (resilient) soft needle cover having an outer (hard) shell and/or the soft needle cover having no shell. The outer shell could be co-moulded or over-moulded or otherwise bonded to the inner soft needle cover. In addition, the outer shell may only cover a part of the soft needle cover, for example a region at the proximal end. The soft inner needle cover could be merely a push or friction fit inside the outer shell.

As shown in FIGS. 1 to 15, the needle cover 23 is a single (resiliently) soft component which has a forward portion 24 (distal end) which does not extend beyond the distal end 50 of the protective shield 19. This provides significant benefits as will be described later. The needle cover 23 comprises the single soft component made of a resilient material which may be an elastomeric polymer or rubber compound to allow the penetration of the sharp tip of the needle, whilst also being compatible with drug contained within the syringe 10. A rearward portion 25 (proximal end) of the cover 23 is disposed within the safety device 8. An opening/bore 26 extends into the cover 23 from the rearward proximal end 25 thereof, the internal distal portion of which is penetrated by the tip of the needle 11 projecting from the nose 12 of the syringe 10. The external surface of the cover 23 has a step 27 in the form of flange distally forward of the end 25, which is arranged to engage a rear facing internal shoulder 42 of the protective shield 19. This engagement of the shoulder 42 with the step 27 securely maintains and retains the cover 23 within the protective shield 19 whilst the protective shield is in the initial (before use) position.

In some embodiments, the needle cover 23 is purely/solely frictionally engaged and/or gripped and/or clasped by the protective shield 19 in order to maintain and hold the needle cover 23 at the first position. In such embodiments, an outer surface of the needle cover 23 does not include any step or flange or any similar features for providing a physical and/or mechanical engagement or abutment between the needle cover 23 and the protective shield 19. This outer surface of the needle cover 23 has a contour that is even/planar and/or substantially smooth and this outer surface is frictionally gripped or clasped by the protective shield 19 when the needle cover is at the first position. Similarly, the protective shield 19 does not include a physical/mechanical feature for the engagement of the needle cover, but instead provides a correspondingly planar and/or substantially smooth inner surface in order to frictionally grip the needle cover 23.

In the illustrated preferred embodiments, the protective shield 19 interlocks the with the needle cover 23. In particular, the present invention provides a mechanism for connecting and/or co-ordinating the function of the different components. Specifically, the protective shield 19 and the needle cover 23 form an interlock which thereby co-ordinates a specific predetermined operational series or sequence of configurations as the protective shield is moved between: (1) a first storage/initial position (before use), (2) an operative/non-shielding (during use) position, and (3) a safety/final shielding position (after use).

The protective shield 19 and the needle cover 23 form an interlock and the relationship is such that the function of the components and the (operational) sequence is set. Initially, in the first step, the interlock is such that the protective shield 19 must first be moved outwardly since the needle cover 23 is not accessible and removable by the user in the initial position. In this initial position, the needle cover 23 is enclosed with the protective shield 19 and may be resiliently urged towards the syringe 10. Access to the needle cover 23 is thereby denied and a user is not able to manually grasp and/or remove the needle cover 23. In the second step, the needle cover 23 is accessible and is removable since the protective shield 19 no longer prevents access to the needle cover 23. In this position, access to the needle cover 23 is now allowed to permit the user to remove the needle cover 23. Accordingly, the protective shield 19 effectively provides a barrier and prevents and/or inhibits access to the needle cover 23 in the initial position, to safeguard the integratory of the drug enclosed within the syringe. Finally, when the injection is complete, the protective shield 19 provides a non-reversible safety position around the needle 11 in the final shielding position after use.

As mentioned above, the protective shield 19 denies access to the needle cover 23 in the initial position to ensure the needle cover remains at the first position until the user is ready to perform an injection, at which point, the user is subsequently provided with access to the needle cover 23 only once the protective shield 19 has been moved to the non-shielding position. In this movement, the engagement between the needle cover 23 and the protective shield 19 is released and any axial compressive forces applied to the needle cover in a proximal direction will now be relaxed.

The present invention thereby provides a sequence of events which are preset and directed and may be co-ordinated by the interlock which defines the functions of the components at the different positions. This sequence must be followed and thereby guides a user through the correct operation of the present invention.

Furthermore, the protective shield 19 provides visual indicators in the form of arrows 45 to indicate how to first commence the operational preset sequence. As shown in FIG. 6, an arrow or two arrows 45 are provided on the protective shield 19 to indicate to a user how to commence the preparation of the injection device in readiness for an injection defined within a strict sequence. A user may instinctively seek or attempt to remove the needle cover 23. However, with the present invention, in the initial position, access to the needle cover 23 is denied. Accordingly, the arrows 45 provide a simple visual instruction or prompt to first move the protective shield 19 outwardly in order to commence the operational injection sequence; to implement the co-ordinating function of the protective shield 19 and the needle cover 23 within their mutual interlocking relationship. This interlock dictates and sets the functional engagement in the various positions of the needle shield in the operational sequence. Accordingly, the arrows 45 provide the initial instructions to first move the protective shield 19 which then enables the needle cover 23 to then be “instinctively grasped” and pulled away from the syringe by the user. This exposes the needle 11 in readiness for performing the injection.

Overall, the protective shield 19 and the needle cover 23 together with the carrier and syringe form an interlock in which the components are intrinsically linked and connected to co-ordinate the function of these components within a predefined sequence of steps when using the invention.

The needle cover could be loosely held within the protective shield and a part of the needle shield in contact with the needle cover (when the cover is at the first position) could be glued and or bonded to a part of the needle cover. So that movement of the shield to a non-shielding position would break/snap/detach the bond. The resultant permanent separation of the two parts would then allow the shield to move freely to a non-shielding position, so as to provide to the user that “a tamper evident seal” had been broken. Which would be confirmatory evidence that the protective shield had not been previously moved from the (before use) initial shielding position.

The movement of the protective shield 19 away from the initial shielding position frictionally disengages the protective shield 19 from the needle cover 23. As it will be appreciated, in some further embodiments, there may be an element of frictional engagement together with a physical/mechanical feature, such as the step/shoulder arrangement of the preferred embodiment.

A rearward face 28 of the cover 23 within the bore 26 has a profile complementary to that of the (bulbous) engagement face 15 of the nose 12 of the syringe 10. The length of the cover 23 and specifically the positional arrangement of the rearward face 28 relative to the step 27 is such that on press-fitting the safety device 8 to the syringe 10, as shown in FIG. 2 and FIG. 9, the rearward face 28 engages with the syringe nose engagement face 15 and is thereby expanded resiliently outwardly into engagement therewith by pushing the carrier of the safety device 8 fully home on the nose 12 of the syringe 10. Thus, when the internal rib of the carrier 21 and 21′is secured to the rear facing shoulder 14 of the hub of the syringe the rearward portion 25 of the cover is subjected to axial compression by the fitting of the safety device 8 to the syringe 10, causing outward radial expansion of the rearward face 28 onto the engagement face 15 of the nose 12 of the syringe to ensure a substantially airtight seal exists or is established between the cover 23 and the nose 12 of the syringe 10, as shown in FIG. 2 and FIG. 9. FIG. 2 shows the syringe 8 has been filled with a drug and the piston and plunger fitted.

The engagement face 15 may be a generally rounded bulbous or a chamfered profile (and/or any other suitable shape) at the nose 12 of the syringe 10 and the rearward face 28 of the cover 23 may have a correspondingly complementary internal profile. These corresponding profiles may provide a substantially airtight hermetic seal between the cover 23 and the syringe 10. The rearward portion 25 of the cover 23 is subjected to a compressive axial force as the safety device 8 is fitted on to the syringe 10 by virtue of the inter-engagement of the step 27 with the internal rear facing shoulder 42 of the protective shield 19 to retain the cover at the first position. Other designs of safety device 8 may be employed, with the cover 23 being modified as appropriate to ensure that the rearward portion 25 of the cover 23 is subjected to a compressive force to establish that the rear end face 28 thereof is in sealing engagement with a corresponding engagement face 15 of the syringe 10, when the safety shield is at an initial position and the cover is at the first position. Designs that generate a compressive force within a soft region of the needle cover when the cover is at the first position, may cause the engagement face 15 of the syringe to outwardly expand the rear end face 28 of (a resilient/soft portion of) the needle cover, or merely cause an end face (for example, a resilient/soft end face) of the needle cover to be in continual abutted compressive contact with a part of the nose/hub end of the syringe, so as to establish and maintain a substantially airtight seal therebetween. In this latter arrangement, a substantially planar or flat face (for example, a resilient/soft end face) of the needle cover 23 may seal against a substantially planar or flat face of the syringe 10 and an ongoing and enduring compressive force maintains an airtight seal therebetween. Similarly, any other cooperating shaped profiles configured to undergo a compressive force (when the safety device 8 is fitted onto a syringe) would also establish and maintain an airtight seal therebetween.

In this position, the sharp tip of the needle 11 is sealed to prevent leakage of the drug by penetrating, to a small extent, into the rubber cover 23. In particular, the sharp tip penetrates into a soft portion of the needle cover 23. This serves to prevent the possibility of drug leakage from the needle 11 and by having only the tip of the needle 11 in the cover 23, the silicon lubricant provided on the shaft of needle 11 to assist easy penetration of the needle through the skin will not be wiped away by the cover 23. Further, the inter-engagement of the cover end face 28 with the syringe nose engagement face 15 also effects a seal which ensures the needle 11 also remains in a sterile condition.

When the syringe 10 and safety device 8 are to be used, the protective shield 19 must first be moved to a non-shielding position to gain access to the needle cover.

The protective shield 19 includes a pair of stub axles 40 which project inwardly. The carrier 18 includes a corresponding pair of holes 41 for rotatably engaging the stub axles 40. The protective shield 19 is therefore rotatably or pivotably movable relative to the carrier 18. In particular, when the carrier 18 (and thence the safety device 8) is attached to the syringe 10, the protective shield 19 is pivotably movable relative to the longitudinal axis of needle 11. The protective shield 19 is initially positioned with a longitudinal axis which is generally parallel to the longitudinal axis of the needle 11 and the protective shield 19 is also generally aligned with the needle 11 in this initial as supplied configuration before use.

The protective shield 19 comprises a partial or incomplete longitudinal tubular sleeve section which circumferentially extends a majority of the way around the circumferential outer surface of the needle cover 23. In this way, a longitudinal gap is formed which enables the semi-tubular sleeve section of the protective shield 19 which accommodates and retains the needle cover at an initial shielding position before use, to be moved outwardly away from the enclosed needle 11. The protective shield 19 has a distal end 50 which provides the outermost or most distal extent of the safety device 8. Specifically, the distal end 50 of the protective shield 19 extends distally at least the same but preferably beyond the distal end 24 of the cover 23. This forward extent of the shield thereby defines the ideal distal dimension for use with standard nest and tub packaging systems. This negates the need for the cover 23 to be manually grasped for removal purposes which may be necessary for prior art devices having telescopic protective shields and which require overly long needle covers projecting past the distal end of the protective shields. This distal dimension of the protective needle shield (with the enclosed needle cover) in the present invention is designed and configured to be no greater than or equivalent to the dimension of standard needle covers used in existing nest and tub systems, such that no modifications need to be made to any of the packaging components used in these standard syringe handling assembly lines to accommodate the safety device 8. As will be described, this is further achieved due to the overall diameter of the safety device 8 of the present invention having a diameter to suit existing standard tray/nest and tub systems. The ability to have a needle shield/carrier (safety device 8) which has a diameter no greater than or equal to or less than the diameter of the syringe 10 together with the ability to have the distal most part of the needle cover 23 at the distal most part of the protective shield 19 or within the length of shield 19, results in the overall assembled length of the safety device 8 being no greater than non-safety standard needle covers disclosed in U.S. Pat. No. 10,973,986 and which are currently used in existing nest and tub systems.

As mentioned above, the protective shield 19 includes an internal shoulder 42 which blocks distal movement of the needle cover 23 away from the first position during assembly of the device 8 onto the syringe 10 and instead urges the cover 23 towards the syringe 10 in the initial position. The present invention advantageously restricts the length of the cover 23 such that the distal most point is defined by the end 50 of the protective shield 19. However, due to this restriction, the needle cover 23 of the present invention cannot be grasped to remove the cover 23 which is required in order to perform the injection. Accordingly, the protective shield 19 is first manipulated manually to move outwardly towards a non-shielding position as shown in FIG. 10. This movement causes the shoulder 42 to disengage from the front face 29 of the step 27, and this movement also releases and or relaxes any urging or compressive forces acting on the cover that was pushing or urging the cover proximally towards the syringe 10.

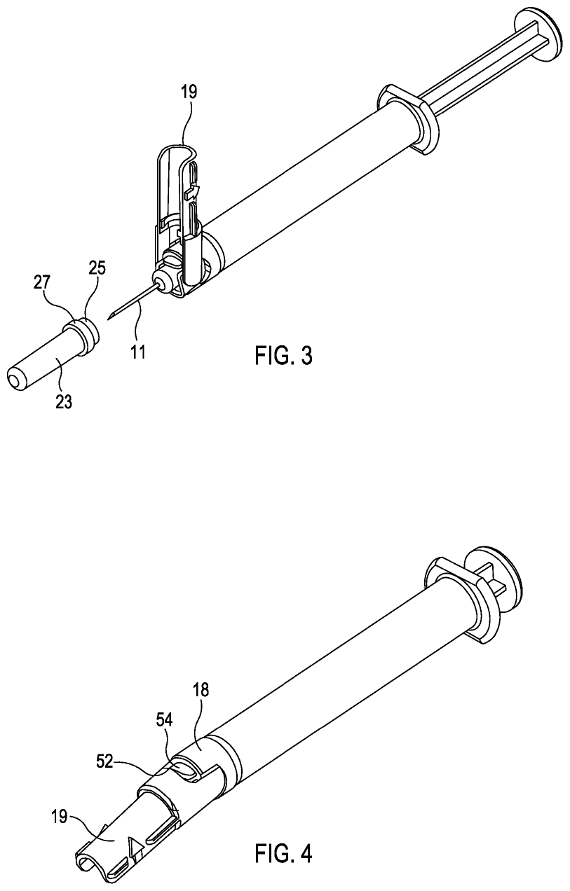

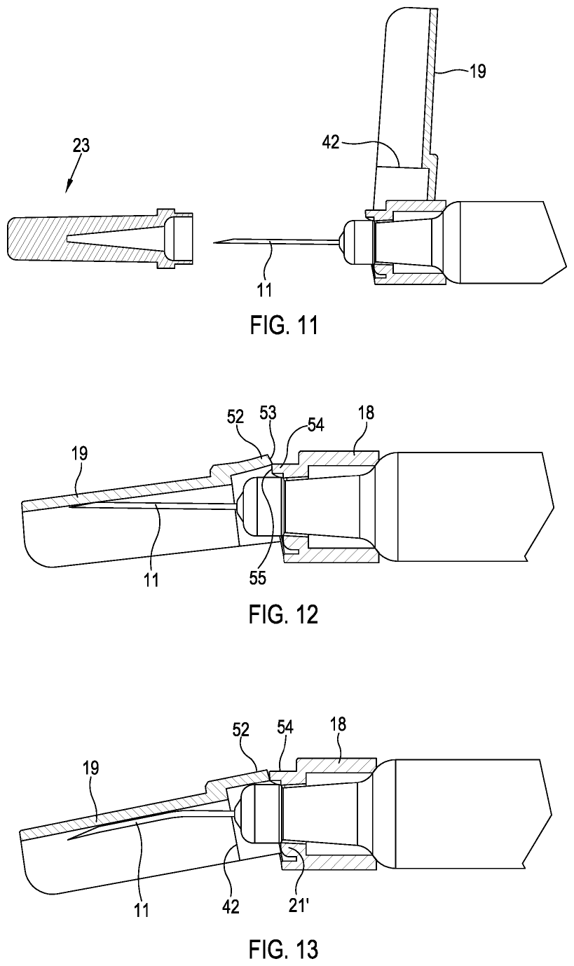

In the outwardly pivoted position shown in FIG. 3 and FIG. 11, the cover 23 is exposed and can be manually grasped and manipulated by a user and withdrawn away from the syringe and needle in a distal direction in readiness to perform the injection. With the protective shield in the non-shielding position, the outer surface of the cover 23 is grasped and pulled distally away from the syringe 10 with the shield in the non-shielding position. The cover 23 is freely moveable in the distal direction since the shoulder 42 of the protective shield 19 is disengaged from the step 27 of the cover 23. The cover 23 can therefore be removed as shown in FIG. 3 and FIG. 11 and can be discarded or placed aside for later disposal, with the syringe and safety device ready for performing an injection.

After the injection has been performed, it is necessary to protect the sharp tip of the needle 11. This is achieved through the inward movement of the protective shield 19 into a locked (after use) final shielding position. In the preferred embodiments, this movement is achieved manually by the intervention of a user to provide what is termed “active protection” of the sharp tip of the needle 11. The protective shield 19 is pivoted inwardly (manually by the user) and an inner surface of the protective shield 19 abuts and contacts the end region of the needle 11, as shown in FIG. 12. The pivotal movement is continued further such that the protective shield 19 deflects/flexes the shaft of the needle 11, as shown in FIG. 4 and FIG. 13. In this position, the longitudinal axis of the protective shield 19 is angled relative to the longitudinal axis of the syringe 10 and carrier 18. This protects and shields the sharp tip of the needle 11 and also prevents re-use of the needle 11. Note that whilst the resilient characteristics of the metallic medical needle tend to resist the inward movement of the shield, this reactive force is very low and is easily overcome by the user as the shield is moved to the final shielding position.

In this final shielding position shown in FIG. 13, the protective shield 19 is locked in the final shielding position relative to the carrier 18 and syringe 10. During the inward pivotal movement of the protective shield, a proximal region 52 of the protective shield 19 resiliently deforms over a distal edge of the carrier 18 provided by a projection (lug) 54 on the carrier, as shown in FIG. 12. The proximal region 52 travels over the projecting lug 54 and eventually passes over the edge of the projection 54. At this point, the resilient properties of the material forming the proximal region 52 may snap back into the original unstressed relaxed state and any deformation of the proximal (abutment) region 52 is then released.

The forward (distal) facing abutment surface 55 of the projecting lug 54 may be angled to maintain the protective shield 19 in the locked position. In particular, the forward facing surface 55 may be angled rearwardly from the outer surface to the inner surface. This angled surface thereby prevents the abutment edge of the resiliently deformable proximal region 52 from being able to travel back over the projecting lug 54 to a non-shielding position, to prevent any attempt to later move the shield away from the final shielding position. In some embodiments, the rearward facing abutment surface 53 of the proximal region 52 of the protective shield 19 may be angled to maintain the protective shield 19 in the locked position. In some embodiments, both the forward facing surface 55 of the projecting lug 54 and the rearward facing abutment surface 53 of the protective shield 19 are angled to maintain the protective shield 19 in the locked position.

The locking of the protective shield 19 in this final shielding position protects the sharp tip of the needle and the angle of the protective shield 19 also provides a visual indication that the safety device 8 has already been used. Furthermore, if the needle 11 shaft has been deformed this may also serve to provide a visual clue prevent the needle 11 from being re-used.

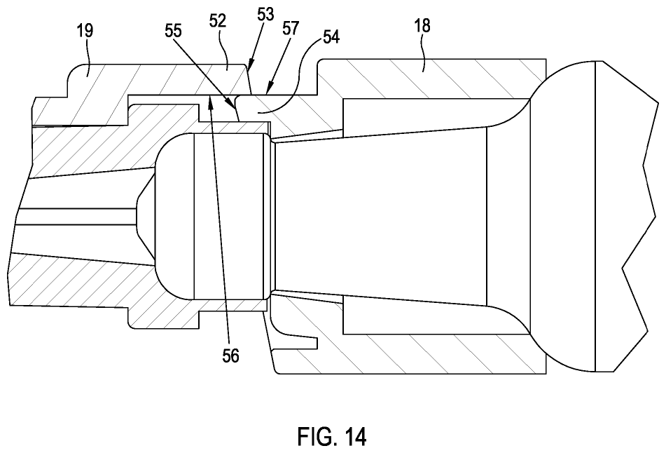

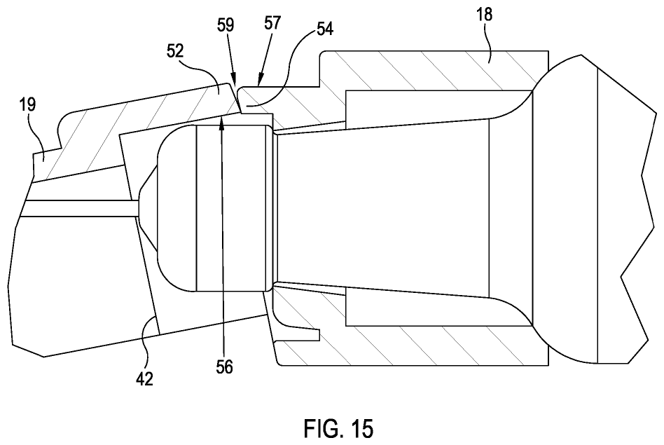

FIG. 14 and FIG. 15 show the locking arrangement of the shield in more detail. The outer surface of the lug 54 comprises a sliding surface 57 over which a part of the protective shield 19 is arranged to move during movement of the shield to the locked position. The inner surface of the protective shield 19 provides a sliding surface 56 in order to aid the sliding movement of the protective shield 19 to the locked final shielding position. In particular, the inner sliding surface 56 of the protective shield 19 is arranged to slide over the outer sliding surface 57 of the lug 54. To move to the locked position, the protective shield 19 is pivoted relative to the carrier 18 which causes the sliding surfaces 56, 57 to move relative to each other such that the sliding surface 56 of the protective shield 19 slides over the sliding surface 57 of the lug 54. The sliding surface 56 of the protective shield 19 (sliding on the sliding surface 57 of the lug 54) then reaches the distal edge of the projection (lug) 54 and the rotational force applied by the user to move the shield to the protecting position, causes a zone within the proximal region 52 of the protective shield 19 to deform outwardly relative to an adjacent zone within the proximal region 52 of the protective shield 19. The deformable zone and the adjacent zone are provided on a continuous (and homogenous) portion of the protective shield 19.

The proximal region 52 of the protective shield 19 provides a deformable zone and comprises resilient/elastic properties. In some embodiments, this zone or region may comprise a reduced or increased thickness (i.e., a relatively thin or thick zone/region) or have a tapered sectional thickness to provide the required deformable properties for this zone/region of the proximal region 52. The protective shield 19 would typically comprise a moulded plastics material or polymer material to provide the required resilient elastic properties. The material may comprise a thermoplastic material having resilient deformable plastic properties which enable the proximal region 52 to deform or flex outwardly during the movement of the protective shield 19 to the locked position and the lug 54 may have a profile shaped to cause said outward deformation or flexing of the region 52. The material and/or the dimensions in this zonal region allow and permit the elastic deformation in order to allow movement the protective shield 19 to the locked final position. As shown in FIG. 15, in the final shielding locked position, the prior deformation of the proximal region 52 has dissipated and the proximal region 52 is in its undeformed and unstressed original relaxed condition or state. In particular, in this locked position, a gap or space 59 may be defined between the contact or abutment faces 53, 55 of the protective shield 19 and the lug 54, as shown in FIG. 15. This demonstrates that the deformation of the proximal region 52 has dissipated and the proximal region 52 and the associated adjacent zones has returned to its original relaxed unstressed condition/state. Furthermore, the (complementary angled) end abutment faces 53, 55 of the protective shield 19 and the lug 54 engage to prevent pivotable return movement of the protective shield 19 towards a position in which the needle 11 may be exposed and present a hazard.

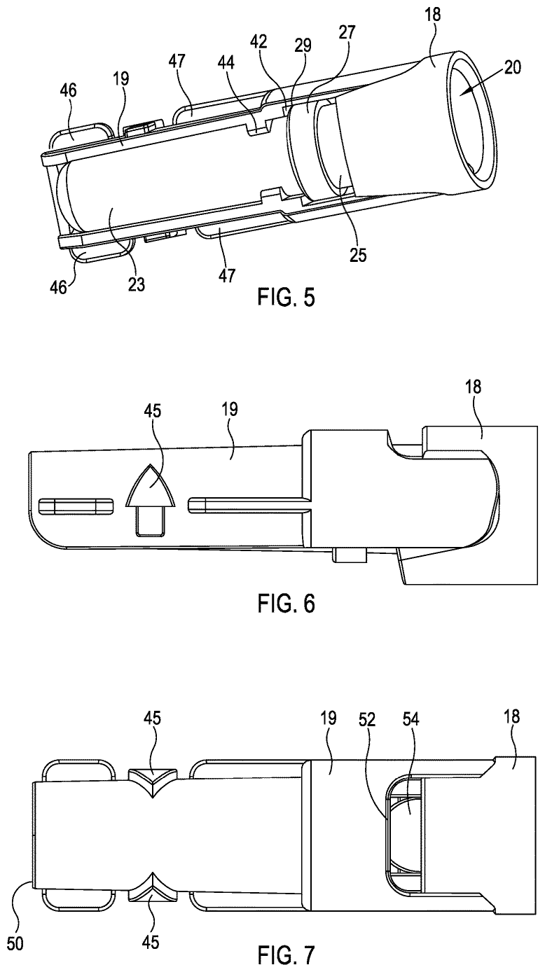

The protective shield 19 may include grip portions 46, 47 on the outer surface (shown adjacent to the arrows 45) to aid the manual manipulation of the protective shield 19, as shown in FIG. 5, FIG. 6 and FIG. 7. These grip portions 46, 47 may comprise ribs extending on the outer surface. The protective shield 19 may have a pair of front or distal ribs 46 and a pair of rear or proximal ribs 47. Each pair providing two ribs 46, 47 offset by approximately 180 degrees around the outer circumference of the protective shield 19. The protective shield 19 also comprises retaining means in the form of retaining clasps 44 to maintain the protective shield 19 in the initial position, as shown in FIG. 5 and FIG. 6. These retaining clasps 44 comprise shaped lugs having inner arcuate surfaces to locate around a part of the outer circumferential surface of the cover 23 to either grip or loosely hold the cover 23. As shown in FIG. 5, the two clasps 44 are spaced apart to provide a release gap therebetween. The extent of this release gap is less than the outer diameter of the cover 23. However, if the cover 23 is made from a resiliently deformable material such that the cover 23 in this area can be deformed sufficiently to enable the cover 23 to pass through the release gap during use, and or the sides of the needle shield will flex outwardly to allow the clasps to travel over the needle cover. The extent of this force is controlled by the size of the release gap and the material of the cover 23 and the shield. In particular, the release force is controlled to be sufficient to prevent any accidental and inadvertent or unwanted movement of the protective shield 19 from this initial position but to allow a user to still move the protective shield 19 to the open position relatively easily albeit with some tactile resistance.

The clasps 44, together with the pivotable coupling/hinge mechanism and shield arrangement, form a sub-assembly comprising the carrier 18, the protective shield 19 and the needle cover 23 and this sub-assembly is maintained together as a unitary assembly and is preferably self-supporting. The creation of the sub-assembly helps with the handling and fitting of the device onto a syringe during manufacture.

Overall, the present invention provides a safety needle device 8 which can be used with standard prefillable syringes within standard nest and tub systems due to the dimensions (length and diameter) of the safety device 8 to replace ordinary non-safety needle cover devices, and importantly the device 8 provides a more secure way of attaching and retaining needle covers on entirely standard prefillable syringes. The protective shield 19 is pivotable from an initial position in which the cover 23 has a proximal portion which is axially compressed during assembly of the device 8 on to the syringe 10 so as to ensure a secure and enduring seal against the nose 12 of the syringe 10. In this initial position, the cover 23 is protected and cannot be accessed or grasped and removed by a user without the user first having to pivot the protective shield 19 outwardly which then exposes the cover 23 to allow a user to then grip and pull off the cover 23 to expose the needle 11 ready for an injection. Once the injection has been completed, the protective shield 19 is pivoted inwardly until it contacts and then flexes the sharp tip of the needle 11 away from its central longitudinal axis (provided the needle is of sufficient length to come into contact with the shield), and continued inward movement of the protective shield 19 then automatically engages a locking means in the final shielding position with the needle 11 and the protective shield 19 being maintained at an angle offset from the longitudinal axis of the syringe 10.

The present invention may be used with pre-filled or pre-fillable glass or plastic syringes. In summary, the present invention provides a safety needle device for a syringe to replace non-safety standard needle covers and which does not require bespoke or modified nest/tray and tub designs and can be integrated into entirely standard tray/nest and tub packaging systems. The present invention also provides an improved way of securely attaching needle covers onto entirely standard syringes to prevent unwanted detachment of the needle cover during changes in the atmospheric pressure encountered during the process of sterilising the syringes prior to filling with a medicament for injection.

A profile at the proximal end of the device 8 is configured (shaped) to engage a profile on the distal end of the needle hub to affect a substantially airtight seal therebetween to maintain the sterility of the medical needle and prevent drug leakage through the needle from within the syringe. The needle cover is located within the protective shield/needle shield, such that when the needle shield is at the initial shielding position the axis of the cover is generally coaxial with the axis of the medical needle. A part of the needle cover and a part of the needle shield are configured to engage when the shield is at the initial shielding position to (or resist distal movement of the needle cover relative to the protective shield) and releasably maintain the needle cover at a first position within the shield to prevent drug leakage and maintain the sterility of the needle during storage of the syringe. The part of the needle shield engaging a part of the needle cover blocks/prevents distal movement of the needle cover relative to the needle hub away from a first position until the needle shield is moved to a non-shielding position. The needle shield is arranged to pivot outwardly (with respect to the axis of the syringe body) and is configured to be manipulated (by a user) away from the initial shielding position to a non-shielding position. This manipulation of the needle shield outwardly away from the initial shielding position, disengages the needle shield from the needle cover in a radially outward arcuate direction and continued movement of the shield to a non-shielding position (then) allows complete detachment of the needle cover from the syringe and needle. Once the injection is completed and the drug has been delivered, the needle shield can be (manipulated) and moved to the (locked) final needle shielding position.

In the present invention, needlestick safety is achieved by using only one extra plastic component as compared to existing non-safety standard needle covers that have a soft inner needle cover and a plastic outer shell and which are used extensively in existing nest and tub systems (as disclosed for example in U.S. Pat. No. 10,973,986). There is a major advantage/importance (cost savings) of using entirely standard type prefillable glass syringes having a typical bulbous needle hub profiles. The present invention utilises “resilient engagement” which may comprise a soft cover or at least a soft part of a needle cover resiliently abutting against an end of the nose (needle hub) of the syringe.

1. A medical needle cover arrangement comprising:
a syringe having a syringe barrel including a needle hub at the distal end thereof, the syringe further comprising
a medical needle having a longitudinal axis and a sharp tip, the medical needle being mounted on the needle hub with the sharp tip projecting distally therefrom;
a safety device comprising a needle shield pivotally coupled to a carrier, wherein the carrier is configured to be secured to the syringe, and the needle shield is configured to move outwardly from an initial shielding position to a non-shielding position;
a needle cover overlying the medical needle to protect the needle and prevent drug leakage from the sharp tip;
the needle cover comprises,
a longitudinal axis,
a profile at a proximal end configured to engage a profile on a distal end of the needle hub to create a substantially airtight seal therebetween to maintain the sterility of the enclosed medical needle within the needle cover when the needle cover located at a first position relative to the needle hub; and
in the initial shielding position, the needle cover is located within the needle shield at said first position, whereat the longitudinal axis of the needle cover is aligned with the longitudinal axis of the needle;
characterised in that:
the needle shield comprises clasping means to clasp and/or grip or loosely hold and retain the needle cover in the first position; and wherein
the needle shield, the carrier and the needle cover are maintained together as a unitary self-supporting safety needle assembly for attachment onto the distal end of the syringe;
whereat a part of the needle cover and a part of the needle shield, are configured to engage when the needle is at the initial shielding position to releasably maintain the needle cover within the needle shield at said first position to prevent drug leakage and maintain the sterility of the medical needle during storage of the syringe;
the part of the needle shield engaging the part of the needle cover blocks distal movement of the needle cover relative to the needle hub distally away from the first position when the safety needle assembly is attached to the distal end of the syringe until the needle shield is moved to the non-shielding position; and wherein
the needle shield is arranged to move outwardly away from the needle cover and is configured to be manipulated away from the initial shielding position to the non-shielding position; said movement of the needle shield away from the initial shielding position disengages the needle shield from the needle cover to thereafter allow subsequent detachment of the needle cover distally away from said first position to uncover the medical needle.

2. A medical cover arrangement according to claim 1 in which the clasping means comprise retaining clasps comprising shaped lugs having inner arcuate surfaces to locate around a part of an outer circumferential surface of the cover to grip or loosely hold the needle cover.

3. A medical needle cover arrangement according to claim 1 in which the clasping means provides a restraining force which must be overcome in order to move the needle shield outwardly from the initial shielding position to the non-shielding position.

4. A medical needle cover arrangement according to claim 1 in which the needle shield comprises a partial cylindrical shield in which a longitudinal gap is provided along a full longitudinal length of the needle shield.

5. A medical needle cover arrangement according to claim 4 in which the clasping means comprises one or more clasps provided on or adjacent to edges of the longitudinal gap of the needle shield.

6. A medical needle cover arrangement according to claim 1 in which the unitary self-supporting safety needle assembly is arranged to be press-fitted onto the syringe.

7. A medical cover arrangement according to claim 1 in which the engagement of the needle shield and needle cover during the attachment of the safety device on to the syringe introduces a compressive axial force in the distal direction in the needle cover, between a rear end of the needle cover and a location where the needle shield engages the needle cover.

8. A medical needle cover arrangement according to claim 1 in which the furthermost distal end of the needle cover is located within the needle shield when the needle shield is in the initial shielding position and the needle cover is in the first position.

9. A medical needle cover arrangement according to claim 1 in which the furthermost distal end of the needle cover projects/protrudes past the furthermost distal end of the needle shield when the needle shield is in the initial shielding position and the needle cover is in the first position.

10. A medical needle cover arrangement according to claim 1 in which the needle shield is secured to the carrier by a hinge mechanism and in which the hinge mechanism defines a fixed rotational axis about which the needle shield rotates relative to the carrier.

11. (canceled)

12. A medical needle cover arrangement according to claim 1 in which the needle shield is arranged to pivot from the initial shielding position to the non-shielding position and in which one of the needle shield or carrier comprises two pivot members which are engaged within two pivot members on the other of the needle shield or carrier.

13. (canceled)

14. A medical needle cover arrangement according to claim 1 in which the needle shield is arranged to pivotally move, between:
the initial shielding position;
the non-shielding position; and
a final shielding position.

15. (canceled)

16. (canceled)

17. A medical needle cover arrangement according to claim 1 in which the needle shield comprises a resiliently deformable locking portion to lock the needle shield relative to the carrier in the final shielding position the locking portion comprising a proximal region of the needle shield, the carrier comprising a locking abutment surface to lock the needle shield relative to the carrier in the final shielding position and the locking surface is defined on a distally extending projection, in which the locking portion is arranged to travel over a part of the locking surface when moving from the non-shielding position to the final shielding position and, in which, the locking portion resiliently deforms as the locking portion moves over the locking surface and in which, in the final shielding position, the locking portion is in an undeformed and relaxed state.

18. (canceled)

19. (canceled)

20. (canceled)

21. (canceled)

22. A medical needle cover arrangement according to claim 1 in which the part of the needle cover comprises a step or outer projection or annular flange.

23. A medical needle cover arrangement according to claim 1 in which the part of the needle shield comprises a rear facing shoulder in order to maintain and hold the needle cover over the needle-at the first position.

24. A medical needle cover arrangement according to claim 1 in which the needle cover is frictionally engaged and gripped by the needle shield, in order to maintain and hold the needle cover at the first position, wherein an outer surface of the needle cover is substantially smooth and this outer surface is frictionally gripped by the needle shield and, in which, the movement of the needle shield away from the initial shielding position frictionally disengages the needle shield from the needle cover.

25. A medical needle cover arrangement according to claim 16 in which the engagement compresses and/or expands a proximal portion of the needle cover.

26. A medical needle cover arrangement according to claim 1 in which the part of the needle shield engages the needle cover at a location spaced forwardly from a rear proximal end thereof when the needle shield is in the initial shielding position to resist movement of the needle cover forwardly and distally away from the syringe hub.

27. (canceled)

28. A medical needle cover arrangement according to claim 1 in which the needle shield extends distally to a position beyond the most distal point of the needle cover such that the entire needle cover is disposed within the longitudinal length of the needle shield.

29. (canceled)

30. (canceled)

31. A method of protecting a medical needle comprising providing a medical needle cover arrangement which comprises:
a syringe having a syringe barrel including a needle hub at the distal end thereof,
the syringe further comprising the medical needle having a longitudinal axis and a sharp tip, the medical needle being mounted on the needle hub with the sharp tip projecting distally therefrom;
a safety device comprising a needle shield pivotally coupled to a carrier, wherein the carrier is configured to be secured to the syringe, and the needle shield is configured to move outwardly from an initial shielding position to a non-shielding position;
a needle cover, overlying the medical needle-to protect the needle-and prevent drug leakage from the sharp tip;
the needle cover comprises,
a longitudinal axis,
a profile at a proximal end configured to engage a profile on a distal end of the needle hub to create a substantially airtight seal therebetween to maintain the sterility of the enclosed medical needle within the needle cover when the needle cover is located at a first position relative to the needle hub;
in the initial shielding position, the needle cover is located within the needle shield at said first position, whereat the longitudinal axis of the needle cover is aligned with the longitudinal axis of the needle; characterised in that:
the needle shield comprises clasping means to clasp and/or grip or loosely hold and retain the needle cover in the first position; and wherein
the needle shield, the carrier and the needle cover are maintained together as a unitary self-supporting safety needle assembly for attachment onto the distal end of the syringe;
whereat a part of the needle cover and a part of the needle shield, are configured to engage when the needle shield at the initial shielding position to releasably maintain the needle cover within the needle shield at said first position to prevent drug leakage and maintain the sterility of the medical needle during storage of the syringe;
the method comprising:
blocking distal movement of the needle cover relative to the needle hub distally away from the first position when the safety needle assembly is attached to the distal end of the syringe until the needle shield is moved to the non-shielding position by engaging the part of the needle shield with the part of the needle cover; and wherein
the needle shield is arranged to move outwardly away from the needle cover and is configured to be manipulated away from the initial shielding position to the non-shielding position; said movement of the needle shield away from the initial shielding position disengages the needle shield from the needle cover to thereafter allow subsequent detachment of the needle cover distally away from said first position to uncover the medical needle.
